(19) United States
(12) Patent Application Publication
(10) Pub. No.: US 2022/0028537 A1
(43) Pub. Date: Jan. 27, 2022

(54) METHOD FOR PROVIDING AN ITEM OF SATISFACTION INFORMATION ABOUT A CUSTOMER'S PREDICTED SATISFACTION WITH REGARD TO A MEDICAL DEVICE

(71) Applicant: Siemens Healthcare GmbH, Erlangen (DE)

(72) Inventors: An NGUYEN, Erlangen (DE); Stefan FOERSTEL, Forchheim (DE); Michael SCHRAPP, Muenchen (DE); Tobias HIPP, Nuernberg (DE); Marie MECKING, Erlangen (DE)

(73) Assignee: Siemens Healthcare GmbH, Erlangen (DE)

(21) Appl. No.: 17/375,306

(22) Filed: Jul. 14, 2021

(51) Int. Cl. G16H 40/40; G16H 40/63; G06N 3/08; G06Q 30/00

(52) U.S. Cl. CPC G16H 40/40; G06Q 30/016; G06N 3/08; G16H 40/63

(57) ABSTRACT

A computer-implemented method is for providing an item of satisfaction information about a customer's predicted satisfaction with regard to a medical device. In an embodiment, the method includes providing input data, the input data including at least one operating parameter of the medical device and at least one item of customer information. The method moreover includes applying a first trained function to the input data, to generate the satisfaction information. The method further includes providing the satisfaction information.

### PRIORITY STATEMENT

The present application hereby claims priority under 35 U.S.C. § 119 to German patent application number DE 102020209200.1 filed Jul. 22, 2020, the entire contents of which are hereby incorporated herein by reference.

### FIELD

Example embodiments of the invention generally relate to a method for providing an item of satisfaction information about a customer's predicted satisfaction with regard to a medical device.

### BACKGROUND

Customer services often have to handle numerous customer inquiries simultaneously. An inquiry may be, for example, a customer telephone call and/or service ticket. In particular, a customer's inquiry may be for example a question about a functionality of a medical device or a report of a breakdown of a medical device or a report of a fault of the medical device etc. In this context, it is frequently necessary to prioritize customer inquiries. For instance, a customer who just has a question about a specific use of the medical device can wait longer for an answer from customer services than a customer whose medical device has completely broken down. In particular, customers with serious problems should be given preferential treatment. Customers with frequently occurring problems should also be given preferential treatment. In particular, prioritization of inquiries is intended to ensure customer satisfaction. In other words, the intention is to ensure that all customers receive the best possible support or assistance. This is intended to ensure customer satisfaction.

Moreover, customer services often have to take suitable action in response to a customer inquiry. For example, customer services may decide that the customer should receive a telephone call. Alternatively, customer services can dispatch a service technician to the customer. The action taken has to be decided based upon the inquiry.

In particular, customer inquiries may be prioritized or the suitable action in response to a customer inquiry determined based upon an item of information about the customer's satisfaction. It is known to determine customer satisfaction based upon customer surveys or social media posts by means of natural language processing (Gräbner et al., “Classification of Customer Reviews based on Sentiment Analysis”, 19th Conference on Information and Communication Technologies in Tourism, 2012; Bagheri et al., “Care more about customers: Unsupervised domain-independent aspect detection for sentiment analysis of customer reviews”, Knowledge-Based Systems, 52, 2013; Genc-Nayebi et al., “A systematic literature review: Opinion mining studies from mobile app store user reviews”, Journal of Systems and Software, 125, 2017). On the other hand, it is known to use a system's log data in order to detect a malicious attack on a system (Kim et al., “Long Short-Term Memory Recurrent Neural Network Classifiers for Intrusion Detection”, International Conference on Platform Technology and Service, 2015; Tuor et al., “Deep Learning for Unsupervised Insider Threat Detection in Structured Cybersecurity Data Streams”, arXiv: 1710:00811v2, 2017) or in order to detect an error in data generated by the system or in the system itself (Min et al., “DeepLog: Anomaly Detection and Diagnosis from System Logs through Deep Learning”, CCS: Computer and Communications Security, 2017; Zhang et al., “Automated IT system failure prediction: A deep learning approach”, IEEE International Conference on Big Data, 2016) or in order to predict maintenance for a specific system component (Sipos et al., “Log-based predictive maintenance”, 20th ACM SIGKDD International Conference on Knowledge Discovery and Data Mining, 2014; US2015/0227838A1).

### SUMMARY

The inventors have discovered that a feature common to all these methods is that just one source of information, for example customer service data (survey data or posts, etc.) or log data etc. of the medical device, is used in order to determine the customer's satisfaction or an item of information about the system of the medical device.

At least one embodiment of the present invention is therefore to provide a method which, based upon log data and customer service data, enables a customer's satisfaction to be determined.

Embodiments of the present invention are directed to a method for providing an item of satisfaction information about a customer's predicted satisfaction with regard to a medical device; a method for providing a first trained function; a system for providing an item of customer satisfaction information with regard to a medical device; a computer program product and a computer-readable storage medium. Advantageous further developments are presented in the claims and in the following description.

The embodiments according to the invention are described below with regard both to the claimed devices or systems and to the claimed method. Features, advantages or alternative embodiments mentioned in this connection are likewise also transferable to the other claimed subjects and vice versa. In other words, the substantive claims (e.g. directed to a device) may also be further developed with the features which are described or claimed in connection with a method. The corresponding functional features of the method are here formed by corresponding substantive modules.

The embodiments according to the invention are moreover described below with regard not only to the claimed method and the claimed systems for providing an item of satisfaction information about a customer's predicted satisfaction with regard to a medical device but also to the claimed method and the claimed systems for training a first trained function. Features, advantages or alternative embodiments mentioned in this connection are likewise also transferable to the other claimed subjects and vice versa. In other words, the method and system claims for training the first trained function may also be further developed with features which are described or claimed in connection with the method and systems for providing an item of satisfaction information about a customer's predicted satisfaction with regard to a medical device and vice versa.

In particular, the method and systems for providing the first trained function may be adapted to the method and systems for providing an item of satisfaction information about a customer's predicted satisfaction with regard to a medical device. Moreover, input data of the method for providing an item of satisfaction information may comprise advantageous features and embodiments of the training input data and vice versa. Moreover, output data of the method for providing an item of satisfaction information may comprise advantageous features and embodiments of the training output data and vice versa.

At least one embodiment of the invention relates to a computer-implemented method for providing an item of satisfaction information about a customer's predicted satisfaction with regard to a medical device. The method comprises the method step of providing input data, the input data comprising at least one operating parameter of the medical device and at least one item of customer information. The method moreover comprises the method step of applying a first trained function to the input data, whereby the satisfaction information is generated. The method moreover comprises the method step of providing the satisfaction information.

In an embodiment, the invention further comprises a computer-implemented method for providing a first trained function. The method comprises the method step of providing training input data, the training input data comprising at least one operating parameter of a medical device and at least one item of customer information. The method moreover comprises the method step of providing training output data, the training output data comprising an item of satisfaction information about the customer's predicted satisfaction with regard to the medical device. The training output data and the training input data here relate to one another. The method moreover comprises the method step of training the first trained function based upon the training input data and the training output data. The method moreover comprises the method step of providing the first trained function.

An embodiment of the invention moreover comprises a system for providing an item of satisfaction information about a customer's predicted satisfaction with regard to a medical device. The system comprises a computing unit and an interface. The computing unit is here configured to provide input data. The input data here comprises at least one operating parameter of the medical device and at least one item of customer information. The computing unit is moreover configured to apply a first trained function, whereby the satisfaction information is generated. The interface is configured to provide the satisfaction information.

Such a system may in particular be configured to carry out the previously described method, and the embodiments and aspects thereof, for providing an item of satisfaction information about a customer's predicted satisfaction with regard to a medical device. The system is configured to carry out this method and the embodiments and aspects thereof by the interface and the computing unit being configured to carry out the corresponding method steps.

An embodiment of the invention also relates to a computer program product with a computer program and to a computer-readable medium. A largely software-based embodiment has the advantage that systems which are already in service can also straightforwardly be retrofitted to operate in the described manner by means of a software update. In addition to the computer program, such a computer program product may comprise additional elements such as for example documentation and/or additional components, as well as hardware components, such as for example hardware keys (dongles etc.) for using the software.

In particular, an embodiment of the invention also relates to a computer program product with a computer program which is directly loadable into a memory of a system having program parts for carrying out all the method steps of an embodiment of the above-described method, and the embodiments and aspects thereof, for providing an item of satisfaction information about a customer's predicted satisfaction with regard to a medical device when the program parts are run by the system.

In particular, an embodiment of the invention also relates to a computer-readable storage medium on which program parts readable and runnable by a system are stored in order to carry out all the method steps of an embodiment of the above-described method, and the embodiments and aspects thereof, for providing an item of satisfaction information about a customer's predicted satisfaction with regard to a medical device when the program parts are run by the system.

An embodiment of the invention moreover relates to a training system for providing a first trained function. The training system comprises a training interface and a training computing unit. The training computing unit is here configured to provide training input data. The training input data here comprises at least one operating parameter of a medical device and at least one item of customer information. The training computing unit is moreover configured to provide training output data. The training output data here comprises an item of satisfaction information about the customer's predicted satisfaction with regard to the medical device. The training output data and the training input data here relate to one another. The training computing unit is moreover configured to train the first trained function based upon the training input data and the training output data. The training interface is here configured to provide the first trained function.

An embodiment of the invention also relates to a training computer program product with a training computer program and to a computer-readable training medium. A largely software-based embodiment has the advantage that training systems which are already in service can also straightforwardly be retrofitted to operate in the manner according to an embodiment of the invention by means of a software update. In addition to the training computer program, such a training computer program product may comprise additional elements such as for example documentation and/or additional components including hardware components, such as for example hardware keys (dongles etc.) for using the software.

In particular, an embodiment of the invention also relates to a training computer program product with a training computer program which is directly loadable into a memory of a system having program parts for carrying out all the method steps of an embodiment of the above-described method, and the embodiments and aspects thereof, for providing a first trained function when the program parts are run by the training system.

In particular, an embodiment of the invention also relates to a computer-readable training storage medium on which program parts readable and runnable by a training system are stored in order to carry out all the method steps of an embodiment of the above-described method, and the embodiments and aspects thereof, for providing a first trained function when the program parts are run by the system.

An embodiment of the invention also relates to a computer-implemented method for providing at least one item of satisfaction information about a predicted satisfaction of a customer regarding to a medical device, the computer-implemented method comprising:

providing input data, the input data including at least one operating parameter of the medical device and at least one item of customer information;

applying a first trained function to the input data, to generate the at least one item of satisfaction information; and

providing the at least one item of satisfaction information.

An embodiment of the invention also relates to a computer-implemented method for providing a first trained function, the computer-implemented method comprising:

providing training input data, the training input data including at least one operating parameter of a medical device and at least one item of customer information;

providing training output data, the training output data including an item of satisfaction information about predicted satisfaction of a customer with regard to the medical device, and the training output data and the training input data relating to one another;

training the first trained function based upon the training input data and the training output data; and

providing the first trained function after the training.

An embodiment of the invention also relates to a system for providing at least one item of satisfaction information about a predicted satisfaction of a customer with regard to a medical device, the system comprising:
at least one processor configured to
   provide input data, the input data including at least one operating parameter of the medical device and at least one item of customer information,
   apply a first trained function to the input data, to generate the at least one item of satisfaction information; and
an interface, configured to provide the at least one item of satisfaction information.

An embodiment of the invention also relates to a non-transitory computer program product storing a computer program, the computer program being directly loadable into a storage device of a system and including program parts for carrying out the method of an embodiment when the program parts are run by the system.

An embodiment of the invention also relates to a non-transitory computer-readable storage medium storing program parts, readable and runnable by a system to carry out the method of an embodiment when the program parts are run by the system.

### BRIEF DESCRIPTION OF THE DRAWINGS

The above-described properties, features and advantages of this invention will be clearer and more readily comprehensible in connection with the following figures and the descriptions thereof. The figures and descriptions are not intended in any way to limit the invention and the embodiments thereof. Identical components in different figures are provided with corresponding reference signs. The figures are not in general true to scale.

In the drawings

FIG. 1 shows a first example embodiment of a method for providing an item of satisfaction information about a customer's predicted satisfaction with regard to a medical device,

FIG. 2 shows a second example embodiment of a method for providing an item of satisfaction information about a customer's predicted satisfaction with regard to a medical device,

FIG. 3 shows a third example embodiment of a method for providing an item of satisfaction information about a customer's predicted satisfaction with regard to a medical device,

FIG. 4 shows an example embodiment of a defined time interval comprising a plurality of disjunctive time blocks and a prediction time block,

FIG. 5 shows an example embodiment of a method for providing a first trained function,

FIG. 6 shows an example embodiment of a training time interval comprising a plurality of disjunctive training time blocks, a prediction training time interval, an escalation time interval and an escalation event,

FIG. 7 shows a system for providing an item of satisfaction information about a customer's predicted satisfaction with regard to a medical device,

FIG. 8 shows a training system for providing a first trained function.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a computer-implemented method for providing an item of satisfaction information about a customer's predicted satisfaction with regard to a medical device. The method comprises the method step of providing input data, the input data comprising at least one operating parameter of the medical device and at least one item of customer information. The method moreover comprises the method step of applying a first trained function to the input data, whereby the satisfaction information is generated. The method moreover comprises the method step of providing the satisfaction information.

The satisfaction information in particular describes the customer's satisfaction for a user. The user may in particular be member of customer services. Customer services may in particular support the medical device and/or advise the customer. The user may here in particular be a service technician or member of service staff or a maintenance technician or a software technician or a member of customer support staff etc. The satisfaction information is here in particular predicted for a period in the future. In other words, the satisfaction information comprises the customer's predicted satisfaction. The customer's satisfaction in particular relates to a medical device. The satisfaction may here in particular relate to a functionality of the medical device and/or to the reliability of the medical device and/or to customer service provision with regard to the medical device etc.

The medical device may in particular comprise a device for clinical laboratory investigations, for example a device for processing or investigating laboratory samples for in vitro tests or a device for laboratory automation. The medical device may in particular be a medical imaging device. The medical imaging device may in particular be an X-ray device and/or a computed tomography (CT) device and/or a magnetic resonance tomography (MRT) device and/or a C-arm and/or a positron-emission tomography (PET) device and/or a single-photon emission computed tomography (SPECT) device and/or an ultrasound imaging device. Alternatively, the medical device may comprise a patient couch and/or a robotic system for assisting an examination and/or operation and/or a software system etc. The software system may in particular be configured to display and/or analyze and/or process medical image data. In particular, the medical device may comprise any possible hardware or software in a medical or clinical context. The medical device may in particular also be a plurality of medical devices or an integrated system of medical devices of the above-stated type. In this manner, the invention can be used for predicting or for providing an item of satisfaction information about a customer's predicted satisfaction with regard to a fleet or an integrated system of devices.

The method step of provision in particular provides the input data for further processing of the input data. Provision of the input data may in particular comprise receiving the input data. The input data may here in particular be provided by the medical device. Alternatively or additionally, the input data may be provided by a customer service system which acquires customer information. Alternatively or additionally, the input data may be provided by a cloud storage system. Alternatively or additionally, the input data may be provided by an internal database. The input data here comprises at least one operating parameter of the medical device and at least one item of customer information.

The operating parameter in particular describes a functionality and/or use and/or an environmental parameter or an environmental condition and/or a performance etc. of the medical device. In particular, the operating parameter describes a technical aspect of the medical device, in particular the operating parameter may comprise an item of information with regard to a type or duration or frequency of use, to a type or duration or frequency of a fault or to a maintenance status, and similar information. Alternatively or additionally, the operating parameter may comprise an item of information about a frequency of an abnormal termination and/or of a restart of a specific process, for example an examination. Alternatively or additionally, the operating parameter may be an item information about a system restart and/or a subsystem restart and/or the frequency thereof. Alternatively or additionally, the operating parameter may comprise an item of information about an external parameter of the medical device. The external parameter may in particular comprise a power supply or power grid stability of the medical device and/or a data network connection or data network stability of the medical device and/or an ambient temperature of the medical device etc. The operating parameter may in particular comprise a numerical value which describes the medical device or the function thereof etc. For example, such a numerical value may describe a number of breakdowns of the medical device. Alternatively or additionally, the operating parameter may comprise an alphabetic value. For example, such an alphabetic value may describe whether a setting of the medical device is “on” or “off”. Alternatively or additionally, the operating parameter may comprise an alphanumeric value. In particular, the alphanumeric value may be a value pair made up of an alphabetic value and a numerical value. For example, the alphanumeric value may comprise a descriptive part, such as “ambient temperature in degrees Celsius” and a value such as “25” for this descriptive part.

The customer information in particular relates to the customers whose satisfaction is to be predicted. In particular, the customer information comprises at least one item of information about the customer. The customer information in particular describes a behavior of the customer and/or a frequency of a customer's attempts to contact customer services and/or a number of medical devices owned by the customer and/or an item of information about spare parts which the customer has already received or ordered etc. The customer information may in particular comprise a numerical value, an alphabetic value and/or an alphanumeric value.

In the method step of applying the first trained function, the satisfaction information is generated by means of the first trained function based upon the input data.

In general, a trained function mimics cognitive functions which people associate with human thinking. In particular, training based on training data can adapt the trained function to new circumstances and recognize and extrapolate patterns.

In general, parameters of a trained function can be adapted by means of training. In particular, supervised training, semi-supervised training, unsupervised training, reinforcement learning and/or active learning may be used for this purpose. Representation learning, which is alternatively known as feature learning, may furthermore be used. In particular, the parameters of the trained functions can be iteratively adapted by a plurality of training steps.

In particular, a trained function may comprise a neural network, a support vector machine, a random tree or a decision tree and/or a Bayesian network and/or the trained function may be based on k-means clustering, Q-learning, genetic algorithms and/or association rules. In particular, a trained function may comprise a combination of a plurality of uncorrelated decision trees or an ensemble of decision trees (random forest). In particular, the trained function can be determined by means of XGBoosting (extreme gradient boosting). In particular, a neural network may be a deep neural network, a convolutional neural network or convolutional deep neural network. A neural network may furthermore also be an adversarial network, a deep adversarial network and/or a generative adversarial network. In particular, a neural network may be a recurrent neural network. In particular, a recurrent neural network may be a network with a long short-term memory (LSTM), in particular a gated recurrent unit (GRU). In particular, a trained function may comprise a combination of the described approaches. In particular, the approaches described here for a trained function are denoted the network architecture of the trained function.

In the method step of providing the satisfaction information, the satisfaction information is provided to the user. Provision of the satisfaction information may in particular comprise displaying the satisfaction information and/or transmitting the satisfaction information, for example via email or SMS, and/or saving the satisfaction information in a storage device or an external database or a cloud storage system.

The inventors have recognized that it is possible to predict a customer's satisfaction. The inventors have recognized that such a prediction may in particular be generated or determined based upon at least one operating parameter and at least one item of customer information. In other words, the inventors have recognized that, based upon a combination of operating data and customer information, the customer's satisfaction can be predicted and provided to the user as satisfaction information. In particular, the inventors have recognized that the customer's satisfaction can be particularly reliably predicted based upon at least one item of technical information from the medical device, the operating parameter, and customer data, the customer information.

According to one embodiment of the invention, the method moreover comprises the method step of determining the at least one operating parameter from log data of the medical device for a first defined time interval.

The log data may in particular comprise a log file. In particular, the log data may comprise an event log file. A log file logs processes which occur in a computer system and/or a network of the medical device. The log file in particular documents processes which occur on the medical device. In particular, the log file may comprise information with regard to the use, functionality, stability etc. of the medical device. Alternatively or additionally, the log data may comprise a stability parameter of the medical device. In particular, the stability parameter may comprise an item of information about an abnormal termination of an image capture and/or about a pop-up and/or about a software update etc. Alternatively or additionally, the log data may comprise at least one environmental parameter or an environmental condition. The environmental parameter may in particular be a temperature, a humidity or a country in which the medical device is located etc.

The first defined time interval may in particular be located in the past. In other words, the first defined time interval may be temporally before a point in time at which the at least one operating parameter is determined. The first defined time interval defines a period for which the at least one operating parameter is determined from the log data. In particular, a time profile of the at least one operating parameter may be determined within the first defined time interval. The first defined time interval may in particular comprise one week or two weeks or three weeks or four weeks or one month or two months or three months or six months, etc. In particular, the first defined time interval may be longer or shorter than the examples listed here. In particular, the first defined time interval may be between two of the listed examples. Defined means in this context that a length or duration of the first defined time interval may be predetermined or defined. In particular, the duration of the first defined time interval may be predefined. Alternatively, the duration of the first defined time interval may be defined by the user. In other words, the user can state or define the time interval for which the at least one operating parameter is to be determined. In particular, the user can define the first defined time interval with the assistance of calendar dates. Alternatively, the user can define the duration in days or weeks or months. In particular, a start of the first defined time interval may be defined from the standpoint of the day on which the user defines the duration of the first defined time interval or on which the operating parameter is determined.

In the method step of determining the at least one operating parameter from the log data, data of relevance to the satisfaction information may in particular be extracted from the log data as the operating parameter. In particular, more than one operating parameter may be determined from the log data.

The inventors have recognized that information in the form of the at least one operating parameter and which may have an influence on the satisfaction information can be determined from the log data. The inventors have moreover recognized that, when the satisfaction information is determined, the log data takes account of the technical aspect of medical device.

According to a further embodiment of the invention, the method moreover comprises the method step of determining the at least one item of customer information based upon sales data and/or customer service data for a second defined time interval.

In particular, the sales data and/or the customer service data may relate to the medical device. In other words, the sales data and/or the customer service data may state information which relates directly or indirectly to the medical device. Information which directly relates to the medical device directly states data which relates to the medical device. Information, which indirectly relates to the medical device states data which for example customers have stated about the medical device. Alternatively, the sales data and/or the customer service data may relate to the customer. In particular, the sales data and/or the customer service data may comprise information about the customer.

In particular, the sales data may comprise information about already supplied or installed spare parts for the medical device. In particular, the sales data may comprise information about ordered spare parts for the medical device. Alternatively or additionally, the sales data may comprise a number of medical devices which the customer owns or are managed or used or administered by customer. Alternatively or additionally, the sales data may comprise the customer's costs. In particular, the customer's costs in relation to the medical device may be stated. In other words, the costs may state how much the customer has spent on the medical device and/or for maintenance work and/or for repair work and/or for spare parts etc. Alternatively or additionally, the costs may state how much the customer has already invested in medical devices which are supported by customer services.

In particular, the customer service data may comprise information about the customer. In particular, the information about the customer may comprise, for example a registered office of the customer or a country of the customer's registered office and/or a time for which the customer has already owned or operated or used a medical device supported by customer services and/or which medical device the customer owns or operates or uses etc. Alternatively or additionally, the customer service data may contain information about one or more of the customer's service tickets. In particular, a customer can create a service ticket if they have a problem with or a question about the medical device. In particular, the service ticket can be sent to customer services. In particular, the customer service data may comprise information about the number of service tickets and/or about an age of a service ticket and/or about a processing status of a service ticket and/or about a type or category of service ticket. In particular, the type or category of a service ticket may describe whether it is a regional or a global service ticket. Alternatively or additionally, the type or category of the service ticket may describe where or by whom the service ticket is being processed. This may in particular comprise information as to whether it is a technical service ticket, a maintenance service ticket, a repair service ticket, a complaint service ticket, a question service ticket etc. Alternatively or additionally, the type or category of the service ticket may describe the escalation level at which the service ticket is located. The escalation level may here be determined by the customer or by the user. The escalation level may here be stated on a discrete or a continuous scale. A high value on the scale may here indicate escalation which has progressed a long way. Alternatively or additionally, the customer service data may comprise information as to how frequently a maintenance or repair technician has made on-site visits to the customer.

The second defined time interval may in particular be located in the past. In other words, the second defined time interval may be temporally before a point in time at which the at least one item of customer information is determined. The second defined time interval defines a period in which the at least one item of customer information is determined from the sales data and/or customer service data. In particular, a time profile of the at least one item of customer information may be determined within the second defined time interval. The second defined time interval may in particular comprise one week or two weeks or three weeks or four weeks or a month or two months or three months or six months, etc. In particular, the second defined time interval may be longer or shorter than the examples listed here. In particular, the second defined time interval may be between two of the listed examples.

In particular, the second defined time interval may differ from the first defined time interval. In particular, the first and the second defined time intervals may be of different length. In particular, the first and the second defined time intervals may be temporally shifted relative to one another. In particular, the first and the second defined time intervals may overlap temporally. In particular, the first and the second defined time intervals may be disjunctive to one another. In other words, the first and the second defined time intervals cannot overlap.

Alternatively, the first and the second defined time intervals may be identical. In other words, the first defined time interval may be equal to the second defined time interval.

Defined means in this context that a length or duration of the second defined time interval may be predetermined or defined. In particular, the duration of the second defined time interval may be predefined. Alternatively, the duration of the second defined time interval may be defined by the user. In other words, the user can state or define the time interval for which the at least one operating parameter is to be determined. In particular, the user can define the second defined time interval with the assistance of calendar dates. Alternatively, the user can define the duration of the second defined time interval in days or weeks or months. In particular, a start of the second defined time interval may be defined from the standpoint of the day on which the user defines the duration of the second defined time interval or on which the operating parameter is determined.

In the method step of determining the at least one item of customer information, the information may be extracted from the sales data and/or the customer service data as customer information which is or might be of relevance to predicting customer satisfaction or for the customer information. In particular, more than one item of customer information may be determined in the method step of determining the at least one item of customer information.

The inventors have recognized that the sales data and/or the customer service data comprise information which is of relevance to the satisfaction information. In particular, the inventors have recognized that information from the sales data and/or customer service data may have an influence on the customer's satisfaction.

According to a further embodiment of the invention, the at least one operating parameter and/or the at least one item of customer information can be determined in the method steps of determining respectively the at least one operating parameter and the at least one item of customer information based upon data from a product lifecycle management (PLM) and/or from a supply chain management (SCM) system.

In particular, product lifecycle management data comprises information which is obtained the during a development process and throughout the entire lifecycle of the medical device. In particular, supply chain management data comprises all the information about a medical device's supply chain. The supply chain in particular starts with manufacture of the medical device and finishes with the installation of the medical device on the customer's premises. The supply chain thus in particular also comprises the transport of the medical device, such as for example shipping of the medical device.

The inventors have recognized that the at least one operating parameter and/or the at least one item of customer information may also be determined from product lifecycle management and/or supply chain management data. Such data may then in particular serve as input data for the first trained function. The inventors have recognized that this data may in particular comprise information which is capable of explaining subsequent breakdowns or problems with the medical device and of predicting an item of satisfaction information. For example, problems during transport or during production may promote a breakdown of specific parts of the medical device. This breakdown in turn has an influence on the satisfaction information with regard to the customer.

According to a further embodiment of the invention, the at least one operating parameter and/or the at least one item of customer information may be determined by a feature extraction algorithm. The feature extraction algorithm here optionally comprises a second trained function.

The at least one operating parameter may here in particular be determined by a first feature extraction algorithm. The at least one item of customer information may here in particular be determined by a second feature extraction algorithm.

The feature extraction algorithm may in particular be configured to extract from the log data and/or sales data and/or customer service data features which, in the form of the operating parameter or the customer information, can influence the satisfaction information. In particular, the at least one operating parameter and/or the at least one item of customer information can be determined by means of the feature extraction algorithm. In particular, the feature extraction algorithm may be adapted to the information which is to be extracted or determined by means of the feature extraction algorithm.

The feature extraction algorithm may here in particular be prepared by an expert. In particular, the expert can define rules according to which the feature extraction algorithm determines the at least one operating parameter and/or the at least one item of customer information. The feature extraction algorithm may alternatively or additionally determine the at least one operating parameter and/or the at least one item of customer information by means of pattern recognition. Alternatively or additionally, the feature extraction algorithm may comprise a count algorithm which counts a specific feature in the log data and/or the sales data and/or the customer service data. In this manner it is, for example, possible to determine the customer's number of service tickets by means of the count algorithm based upon the customer service data. Alternatively or additionally, it is, for example, possible to determine a number of abnormal scan terminations from the log data.

In particular, the feature extraction algorithm may comprise a second trained function. For this purpose, the second trained function may be trained, for example automatically, on the log data to determine the at least one operating parameter. In particular, the feature extraction algorithm for determining the at least one operating parameter may comprise a sequence recognition algorithm (sequence mining or sequence pattern mining). Patterns in partially structured data may be recognized in this manner. The second trained function may in particular comprise “natural language processing” for analyzing text data or alphabetic data or alphanumeric data for determining the at least one item of customer information.

In particular, the feature extraction algorithm may comprise a combination of the described functions or algorithms.

The feature extraction algorithm may in particular access the log data and/or the sales data and/or the customer service data by means of a Python API.

The feature extraction algorithm may in particular comprise data preprocessing. The data may in particular comprise the log data and/or the sales data and/or the customer service data and/or the at least one operating parameter and/or the at least one item of customer information. By means of preprocessing, the at least one operating parameter and/or the at least one item of customer information is processed in particular in such a manner that it is suitable as input data for the first trained function. In particular, by means of preprocessing, the log data and/or the sales data and/or the customer service data may be processed in such a manner that the at least one operating parameter or the at least one item of customer information may be determined therefrom.

The inventors have recognized that the at least one operating parameter and/or the at least one item of customer information can be automatically determined by means of the feature extraction algorithm. In particular, the inventors have recognized that, using the feature extraction algorithm, the at least one operating parameter and/or the at least one item of customer information may be preprocessed in such a manner that it is suitable as input data for the first trained function.

According to a further embodiment of the invention, the first and/or second time interval comprises a plurality of disjunctive time blocks. The disjunctive time blocks here follow one another temporally. The at least one operating parameter or the at least one item of customer information is here determined cumulatively for each of the time blocks.

In particular, a time block may comprise a temporal subportion or a temporal interval or an interval of time of the first and/or second defined time interval. In particular, disjunctive means that the time blocks of a defined time interval are not superimposed or do not overlap temporally. In particular, the disjunctive time blocks of a defined time interval may directly follow one another temporally. In other words, the time blocks of a defined time interval may follow one another without gaps. In particular, the time blocks of the plurality of disjunctive time blocks may be of equal size or length. In other words, the time blocks may have the same duration. Alternatively, the time blocks may be of differing size or length. In particular, the first defined time interval may be subdivided into a first plurality of disjunctive time blocks. In particular, the second defined time interval may be subdivided into a second plurality of disjunctive time blocks. In particular, the first plurality of the disjunctive time blocks may correspond to the second plurality of disjunctive time blocks. In particular, the number of disjunctive time blocks for the first and/or second defined time interval is predetermined by the length or duration of the corresponding first and/or second defined time interval and/or by the length or duration of the time blocks. In particular, the first and/or second defined time interval may comprise one time block. Alternatively, the first and/or second defined time interval may comprise more than one time block.

In particular, a time block may for example comprise a week or a month.

In particular, “cumulatively” means that the at least one operating parameter or the at least one item of customer information data comprises data about the complete time block. In particular, this may mean that the data about the time block is acquired in time-averaged manner, or that the data about the time block is summed, or that the data about the time block is for example collected in a list etc. In other words, a time profile of the at least one operating parameter or of the at least one item of customer information is determined in temporal steps having the size or duration of a time block.

The inventors have recognized that fluctuations can be offset by cumulating the at least one operating parameter or the at least one item of customer information. For example, in the case of a time block which comprises one week, a fluctuation of the at least one operating parameter or of the at least one item of customer information can be offset by the weekend. The inventors have recognized that, by subdividing the first and/or second defined time interval into disjunctive time blocks, it is possible to determine a time profile of the at least one operating parameter or of the at least one item of customer information. The inventors have moreover recognized that the time profile may serve as input data for the first trained function for determining the satisfaction information and that this leads to an improvement in the predicted customer satisfaction or the satisfaction information.

According to a further embodiment of the invention, the satisfaction information is generated for at least one prediction time block. The at least one prediction time block here temporally follows the first and/or second defined time interval.

In particular, the prediction time block may comprise a day or a week or a month etc. In particular, the satisfaction information or the customer's predicted satisfaction with regard to the medical device may be ascertained within the prediction time block. In particular, the prediction time block may be located in the future at a point in time of determining the satisfaction information.

In particular, the satisfaction information may be determined for a plurality of disjunctive prediction time blocks. In particular, the disjunctive prediction time blocks may temporally follow one another. In particular, an item of satisfaction information may be generated for each of the disjunctive time blocks. In particular, a time profile of the customer's satisfaction can be predicted in this way.

The inventors have recognized that providing the satisfaction information for at least one prediction time block gives the user a feel for how the customer's satisfaction is developing over time. Moreover, the user can in this manner estimate how much time they have to respond and satisfy the customer.

According to a further embodiment of the invention, the satisfaction information comprises at least one item of classification information.

In particular, the customer's satisfaction may be classified by means of the classification information. In particular, the classification information indicates a measure of the customer's satisfaction with regard to the medical device.

In particular, the customer's satisfaction may be classified into discrete classes. In other words, the classification information indicates an assignment of the customer's satisfaction into one class of the discrete classes. In other words, the classification information indicates the class to which the customer's satisfaction has been or is being assigned. For example, the customer's satisfaction can be divided into or assigned to four classes. Assignment to class “1” may here mean that the customer is very satisfied and has no complaints. Assignment to class “4” may indicate a maximum escalation level. In other words, a customer whose satisfaction information comprises a class “4” item of classification information is very dissatisfied. Alternative divisions into classes are possible. In particular, classification may be into more or less than four classes. Alternatively, the highest class, for example class “4”, may indicate that the customer is very satisfied while class “1” indicates the highest escalation level. In particular, the predicted customer satisfaction may be classified similarly to a school grading scheme. In particular, the predicted satisfaction may be divided into two classes with “0” meaning that the customer is satisfied and “1” meaning that the customer is very dissatisfied. Alternatively, the meanings of “0” and “1” can be swapped. Alternatively, the discrete classes can be designated not with numbers but instead with words. For example, the customer's satisfaction can be described symbolically by means of a temperature scale. For this purpose, the classes may for example be designated as follows: “cold”, “lukewarm”, “warm” and “hot”. “Cold” here means that the customer is satisfied and “hot” that the customer is very dissatisfied and the highest escalation level has been reached.

Alternatively, the classification information may comprise an indication of the customer's satisfaction along a continuous scale. In particular, the scale comprises a plurality of continuous classes. In particular, the scale may comprise values between 1 and 10. In particular, the classification information may assume any desired value between 1 and 10. In particular, the classification information may comprise the value between 1 and 10 which describes the customer's satisfaction. In particular, a value of “1” may mean that the customer is very satisfied and a value of “10” that the customer is very dissatisfied. The values between 1 and 10 describe gradations of the customer's satisfaction between the two limit values. Alternatively, the meanings of “1” and “10” can be swapped. Alternatively, limit values other than 1 and 10 are also conceivable for the continuous scale. Alternatively, the limit values of the continuous scale may be “0” and “1”. The customer's satisfaction is here stated as a probability for escalation or for major dissatisfaction of the customer.

The inventors have recognized that it is possible by means of the classification information simply and clearly to provide the user with an indication of the customer's satisfaction. In particular the inventors have recognized that the user can straightforwardly derive actions to improve or ensure the customer's satisfaction from the classification information.

According to a further embodiment of the invention, the satisfaction information comprises at least one item of explanatory information about the at least one item of classification information.

In particular, the explanatory information comprises a reason or a clarification or an explanation as to why the customer's satisfaction was assigned the class stated in the classification information. In particular, the explanatory information may state which items of input data (operating parameter and/or customer information) were crucial to the assignment to the class stated in the classification information. In other words, the explanatory information comprises an item of information about how the classification information came about. For example, the number of service tickets in a specific period may be crucial to assigning the customer's satisfaction to a specific class.

If the first trained function comprises a random tree or decision tree or an ensemble of decision trees (random forest) or a XGBoost, the explanatory information may be determined by means of a tree explainer algorithm. If the first trained function comprises a (deep) neural network, for example a recurrent neural network and/or convolutional neural network and/or a long short-term memory and/or a gated recurrent unit, the explanatory information may in particular be determined by means of a sensitivity attention mechanism and/or by means of a relevance propagation approach and or by means of a deep explainer.

The inventors have recognized that the explanatory information enables the user to understand why they have received a specific item of classification information for the customer. The user can conclude therefrom whether the classification information is reasonable and what action they must or should take to improve and/or ensure the customer's satisfaction.

According to a further embodiment of the invention, the method further comprises the method step of providing the satisfaction information in a decision support system and the method step of the decision support system deriving a recommended action from the satisfaction information.

In particular, provision of the satisfaction information may comprise the decision support system displaying the satisfaction information. In particular, the display may take the form of a graphic or image and/or text on an output medium. The output medium may in particular be a screen or a computer screen. In particular, the decision support system may comprise the output medium. In particular, the decision support system may comprise a graphical user interface (GUI). In particular, the satisfaction information may be provided by means of the GUI. Alternatively, provision may also comprise transmission of a message, in particular an email and/or a text message (SMS), to the user.

The recommended action may in particular describe what measure or action or type of action the user should take or carry out in order to ensure or improve the customer's satisfaction or in order to prevent escalation by the customer. The recommended action may be for example contacting the customer by telephone or email and/or visiting the customer and/or making an offer to the customer (e.g. time-limited free-of-charge use of a software add-on etc.) and/or priority treatment of the customer and/or a deadline within which the customer should be contacted at the latest etc. Alternatively or additionally, the recommended action may state what problem the customer has or whether it is a technical problem or a service problem. Alternatively or additionally, the recommended action may comprise prioritizing a plurality of customers whose satisfaction information is provided. In other words, the recommended action may output a recommendation as to which customer the user should focus on.

In particular, the recommended action may be derived by the decision support system based upon the satisfaction information. In particular, the decision support system can derive an urgency of the recommended action based upon the classification information. In particular, the decision support system can derive the type of action based upon the explanatory information. In particular, the recommended action can be provided in the decision support system. In particular, the recommended action may be displayed. In particular, the recommended action may be displayed together with the satisfaction information. In particular, the recommended action may be provided or displayed by means of the GUI of the decision support system.

In alternative embodiments, the user may themselves derive the recommended action based upon the satisfaction information.

The inventors have recognized that providing the satisfaction information to the user permits targeted action in order to assure or ensure or improve the customer's satisfaction. The inventors have moreover recognized that deriving the recommended action by way of the decision support system assists the user in making a rapid decision as to when which action is necessary or recommended in order to assure the customer's satisfaction.

In an embodiment, the invention further comprises a computer-implemented method for providing a first trained function. The method comprises the method step of providing training input data, the training input data comprising at least one operating parameter of a medical device and at least one item of customer information. The method moreover comprises the method step of providing training output data, the training output data comprising an item of satisfaction information about the customer's predicted satisfaction with regard to the medical device. The training output data and the training input data here relate to one another. The method moreover comprises the method step of training the first trained function based upon the training input data and the training output data. The method moreover comprises the method step of providing the first trained function.

In particular, the training output data may be prepared by an expert or a user. In particular, the training input data and the training output data relate to a period in the past. In particular, the expert or user is already aware of the customer's satisfaction with regard to the training input data for creating the training output data. In particular, the training output data may be derived by the expert or the user from the training input data. In particular, the training input data and the training output data thus relate to one another.

In particular, in the method step of training the first trained function, training may proceed by means of supervised training or unsupervised training. In particular, supervised learning may comprise random over- or undersampling or synthetic minority oversampling. In particular, any imbalance of the training input data and training output data with regard to an item of classification information of the training output data may be offset as a consequence. The classification information of the training output data is configured similarly to the classification information of the satisfaction information. In particular, supervised learning may alternatively or additionally comprise cost-sensitive learning. In particular, cost-sensitive learning can more strongly weight an underrepresented class of the classification information of the training output data during training. Unsupervised learning may in particular comprise anomaly detection with a deep autoencoder model.

The inventors have recognized that data from the past can be used as training input data. In particular, the inventors have recognized that the training output data for the past may be prepared by an expert or user and be based on the customer's actual satisfaction in the past.

According to an optional embodiment of the invention, the at least one operating parameter is determined for a first training time interval and the at least one item of customer information for at least one second training time interval. The first training time interval and the second training time interval here comprise a plurality of disjunctive training time blocks. The training output data here comprises the satisfaction information for at least one prediction training time block. The prediction training time block here temporally follows the first and/or second training time interval.

The first and the second training time intervals may be configured similarly to the first and the second defined time intervals. In particular, the disjunctive training time blocks of the first and the second training time intervals may also be configured similarly to the disjunctive time blocks of the first and the second defined time intervals. The prediction training time block may be configured similarly to the prediction time block. The prediction training time block is, however, located in the past. In particular, the customer's satisfaction is known within the prediction training time block.

The inventors have recognized that a similar temporal description of the input data and output data for the training and the method for determining the satisfaction information enables maximally efficient training which is adapted to the data.

According to a further embodiment of the invention, the training input data is acquired outside an escalation time interval. The escalation time interval is here initiated by an escalation event.

In particular, the escalation event may here for example be a complaint email from the customer to customer services and/or a telephone call to customer services and/or a threat of consequences (e.g. contract termination) by the customer and/or a customer changing to another supplier etc. In particular, the escalation event or the start of the escalation time interval can be defined manually. Alternatively, the escalation event or the start of the escalation time interval can be defined automatically. In particular, in this case the escalation time interval is the time interval or the period or the interval of time which is still influenced by the escalation event. In particular, the escalation event may be flagged by an expert or a user. In particular, the escalation time interval directly follows the escalation event. In particular, the escalation time interval may comprise one week or two weeks or three weeks or a month after the escalation event. In other words, the escalation time interval may comprise one week or two weeks or three weeks or a month after the escalation event. In particular, the duration may also comprise a value between or greater or less than the listed values. In particular, the duration of the escalation time interval may also depend on the type of escalation event. For example, a customer contract change may initiate a longer escalation time interval than a complaint email. In particular, the duration of the escalation time interval may be defined or determined by the expert or the user.

In particular, the training input data is acquired in such a manner that it is not influenced by an escalation event, i.e. is located outside the escalation time interval. In particular, the training output data is also acquired or determined in such a manner that it is located outside the escalation time interval.

The inventors have recognized that an escalation event may influence the training of the first trained function such that independent analysis of the input data by the first trained function may optionally not be possible. The inventors have recognized that data which is located temporally outside an escalation time interval is preferably used for training the first trained function.

According to a further embodiment of the invention, the first trained function trained according to the described method of the invention may be used for providing the satisfaction information.

According to a further embodiment of the invention, the first trained function is continuously further trained by means of feedback. The feedback is here based on a match value between the provided satisfaction information and an ascertained customer satisfaction.

In particular, the user may subsequently determine or ascertain the customer's ascertained or actual satisfaction. The ascertained customer satisfaction may be ascertained once the prediction time block for the satisfaction information has elapsed, for example with the assistance of the user's experience or feedback from the customer. The user's experience may for example comprise a contract extension or a contract termination or the user's assessment.

In particular, the match value may then be determined by comparing the provided satisfaction information of the predicted satisfaction with the ascertained customer satisfaction. In particular, the match value may be determined by the expert and/or the user. In particular, the match value may comprise a value on a continuous scale or a discrete class. For example, the match value may comprise classes similar to a school grading scheme. In particular, the match value may comprise a class “1” when there is a very good match and a class “6” when there is no match.

The inventors have recognized that the first trained function can be continuously improved and adapted in this manner. The inventors have moreover recognized that the assessment of the predicted customer satisfaction and the objectivity of this assessment can also be improved as a consequence.

According to a further embodiment of the invention, the first trained function is selected from a plurality of first trained functions. The selection is here based on the match value.

This method is in particular known as “model selection”.

In particular, selection may proceed during the training of the first trained function. In particular, a plurality of first trained functions may be trained during the training. In particular, the first trained functions may differ with regard to their functionality or network architecture. Examples of network architectures are described above. In particular, the match value may be determined during the training. The match value is here determined based upon the training output data and the satisfaction information predicted by the first trained function. In other words, the training output data is compared during the training with the satisfaction information determined by the first function. The match value may be determined from this comparison. The match value may be configured as described above. The match value may in particular be determined automatically or manually. In particular, the first trained function which is selected may be the one whose ascertained satisfaction information has the best match value with the training output data.

In particular, the first trained function may alternatively or additionally be selected while the method according to the invention is being carried out. In particular, the satisfaction information may be determined in parallel for each of the first trained functions. The selected first trained function is here the only one to provide the user with the satisfaction information. The match value may be determined as described above for each of the first trained functions by feedback from the user. Based upon the match value, it is possible the next time or in the event of the method being carried out repeatedly to provide the satisfaction information which was ascertained by the first trained function with the best match value. In other words, the selected first trained function may be replaced by another first trained function if, according to the feedback, its match value is better than that of the originally selected first trained function. The selection may alternatively be based on an average of a plurality of match values for a plurality of items of satisfaction information. In particular, it is possible to check continuously which first trained function is most suitable or has the best match value.

The inventors have recognized that it is possible by means of “model selection” flexibly to select the most suitable first trained function for predicting the customer's satisfaction or for ascertaining the satisfaction information. In this manner, it is possible to ensure that the satisfaction information which is most suitable with regard to the match value is provided.

An embodiment of the invention moreover comprises a system for providing an item of satisfaction information about a customer's predicted satisfaction with regard to a medical device. The system comprises a computing unit and an interface. The computing unit is here configured to provide input data. The input data here comprises at least one operating parameter of the medical device and at least one item of customer information. The computing unit is moreover configured to apply a first trained function, whereby the satisfaction information is generated. The interface is configured to provide the satisfaction information.

Such a system may in particular be configured to carry out the previously described method, and the embodiments and aspects thereof, for providing an item of satisfaction information about a customer's predicted satisfaction with regard to a medical device. The system is configured to carry out this method and the embodiments and aspects thereof by the interface and the computing unit being configured to carry out the corresponding method steps.

An embodiment of the invention also relates to a computer program product with a computer program and to a computer-readable medium. A largely software-based embodiment has the advantage that systems which are already in service can also straightforwardly be retrofitted to operate in the described manner by means of a software update. In addition to the computer program, such a computer program product may comprise additional elements such as for example documentation and/or additional components, as well as hardware components, such as for example hardware keys (dongles etc.) for using the software.

In particular, an embodiment of the invention also relates to a computer program product with a computer program which is directly loadable into a memory of a system having program parts for carrying out all the method steps of an embodiment of the above-described method, and the embodiments and aspects thereof, for providing an item of satisfaction information about a customer's predicted satisfaction with regard to a medical device when the program parts are run by the system.

In particular, an embodiment of the invention also relates to a computer-readable storage medium on which program parts readable and runnable by a system are stored in order to carry out all the method steps of an embodiment of the above-described method, and the embodiments and aspects thereof, for providing an item of satisfaction information about a customer's predicted satisfaction with regard to a medical device when the program parts are run by the system.

An embodiment of the invention moreover relates to a training system for providing a first trained function. The training system comprises a training interface and a training computing unit. The training computing unit is here configured to provide training input data. The training input data here comprises at least one operating parameter of a medical device and at least one item of customer information. The training computing unit is moreover configured to provide training output data. The training output data here comprises an item of satisfaction information about the customer's predicted satisfaction with regard to the medical device. The training output data and the training input data here relate to one another. The training computing unit is moreover configured to train the first trained function based upon the training input data and the training output data. The training interface is here configured to provide the first trained function.

An embodiment of the invention also relates to a training computer program product with a training computer program and to a computer-readable training medium. A largely software-based embodiment has the advantage that training systems which are already in service can also straightforwardly be retrofitted to operate in the manner according to an embodiment of the invention by means of a software update. In addition to the training computer program, such a training computer program product may comprise additional elements such as for example documentation and/or additional components including hardware components, such as for example hardware keys (dongles etc.) for using the software.

In particular, an embodiment of the invention also relates to a training computer program product with a training computer program which is directly loadable into a memory of a system having program parts for carrying out all the method steps of an embodiment of the above-described method, and the embodiments and aspects thereof, for providing a first trained function when the program parts are run by the training system.

In particular, an embodiment of the invention also relates to a computer-readable training storage medium on which program parts readable and runnable by a training system are stored in order to carry out all the method steps of an embodiment of the above-described method, and the embodiments and aspects thereof, for providing a first trained function when the program parts are run by the training system.

FIG. 1 shows a first example embodiment of a method for providing an item of satisfaction information about a customer's predicted satisfaction with regard to a medical device.

In the method step PROV-01 of providing input data, input data for determining the satisfaction information is received from a system SYS for providing the satisfaction information. The data may here be sent to the system SYS by the medical device and/or by a customer service system. Alternatively, in the method step PROV-01 of providing the input data, the input data may be retrieved by the system SYS. In particular, the input data may be retrieved or provided from an internal database of the system SYS and/or from an external database. The external database may in particular be stored on a cloud storage system and/or a server system. Alternatively, in the method step PROV-01 of providing the input data, the input data may in particular be determined or acquired by the system SYS.

The input data comprises at least one operating parameter of the medical device and at least one item of customer information.

The medical device may in particular comprise a device for clinical laboratory investigations, for example a device for processing or investigating laboratory samples for in vitro tests or a device for laboratory automation. The medical device may in particular be a medical imaging device. The medical device may in particular be an X-ray device or a computed tomography (CT) device or a magnetic resonance tomography (MRT) device or a C-arm or a positron-emission tomography (PET) device or a single-photon emission computed tomography (SPECT) device or an ultrasound imaging device. Alternatively, the medical device may be a patient couch or a robotic system or a software system. The software system may in particular be configured to display and/or analyze and/or process medical image data. In particular, the medical device may comprise any possible hardware or software in a medical or clinical context. The medical device may in particular also be a plurality of medical devices or an integrated system of medical devices of the above-stated type. In this manner, the invention can be used for predicting or for providing an item of satisfaction information about a customer's predicted satisfaction with regard to a fleet or an integrated system of devices.

The operating parameter describes for example the use and/or an environmental parameter or an environmental condition and/or the performance and/or a functionality of the medical device. Use may for example describe how often which program or function of the medical device is used. Use may moreover describe the capacity utilization of the medical device. Performance may state a measure of the efficiency of the medical device. Efficiency may in particular describe a duration which the medical device requires for running a program. Functionality may in particular describe whether all the components of the medical device are functioning as intended. The environmental parameter may in particular comprise a room temperature and/or a device temperature and/or an atmospheric humidity and/or a country in which the medical device is located, etc. In particular, the operating parameter may comprise an item of information with regard to a type or duration or frequency of use, to a type or duration or frequency of a fault or to a maintenance status, and similar information.

Alternatively or additionally, the operating parameter may comprise an item of information about a frequency of an abnormal termination and/or of a restart of a specific process, for example an examination. Alternatively or additionally, the operating parameter may be an item information about a system restart and/or a subsystem restart and/or the frequency thereof. Alternatively or additionally, the operating parameter may comprise an item of information about an external parameter of the medical device. The external parameters may in particular comprise a power supply or power grid stability of the medical device and/or a data network connection or data network stability of the medical device etc. The operating parameter may comprise a numerical value or an alphanumeric value or an alphabetic value.

The customer information may in particular comprise a behavior of the customer and/or a frequency or number of a customer's attempts to contact customer services and/or a number of medical devices owned or managed by the customer. In other words, the customer information may comprise any information about the customer whose satisfaction information is to be provided. The customer information may comprise a numerical value or an alphanumeric value or an alphabetic value.

In the method step APP of applying the first trained function, the satisfaction information is determined or generated from the input data. The satisfaction information describes the customer's predicted satisfaction for a period, a “prediction time block” VZB in the future. The satisfaction information is here based on the at least one operating parameter and the at least one item of customer information. The satisfaction information comprises at least one item of classification information. The classification information describes the customer's predicted satisfaction with the assistance of a discrete or continuous scale or classification. For example, a customer who is predicted to be very satisfied may be assigned the classification information “1”. A customer who is predicted to be very dissatisfied may be assigned the classification information “4”. Alternatively, “4” may for example denote very satisfied and “1” very dissatisfied. The gradations between these classes may be discrete or continuous. The classes may alternatively be named, for example “satisfied” to “very dissatisfied”. The classes may alternatively be classified according to the principle of a school grading scheme. The satisfaction information may moreover comprise an item of explanatory information about the classification information. The explanatory information describes how the classification information came about. In other words, the explanatory information provides a reason which the customer's satisfaction was predicted according to the classification information. The explanatory information indicates which of the input data was crucial to the corresponding classification of the classification information.

In the method step PROV-02 of providing the satisfaction information, the satisfaction information generated in the method step APP of applying the first trained function is provided to a user. The user may in particular be a member of customer services staff. Customer services may in particular be tasked with maintaining the medical device or with organizing the maintenance of the medical device and/or with supporting the customer. The satisfaction information may be provided by display of the satisfaction information on a display medium or output medium, for example a screen. Alternatively, the satisfaction information may be provided in this method step by means of transmitting the satisfaction information to the customer, for example by SMS or email.

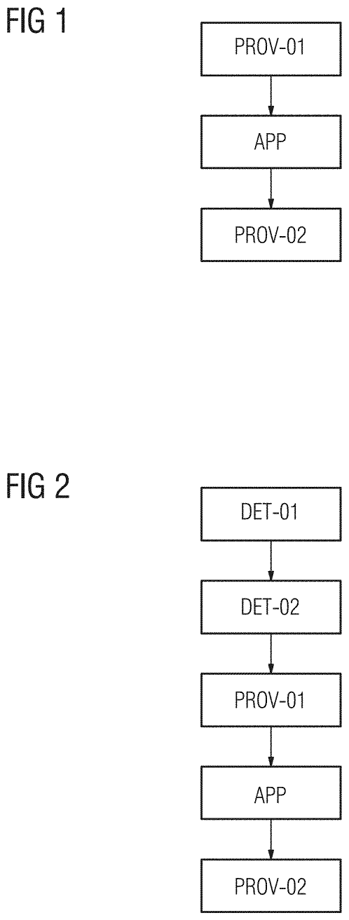

FIG. 2 shows a second example embodiment of a method for providing an item of satisfaction information about a customer's predicted satisfaction with regard to a medical device.

The method steps PROV-01 of providing the input data, APP of applying the first trained function and PROV-02 of providing the satisfaction information are carried out in accordance with the description in relation to FIG. 1.

In the method step DET-01 of determining the at least one operating parameter, the at least one operating parameter is determined from log data of the medical device for a first defined time interval ZS.

The log data may in particular comprise at least one log file and/or an event log file of the medical device. The log data may for example comprise information which describes how (which function, how often, for how long) the medical device is used, whether all the components of the medical device are functioning as intended, or which parameters (e.g. displacement parameters of a patient couch and/or a robot arm, exposure time, X-ray voltage, etc.) are set. The log data may alternatively or additionally comprise information about an environmental parameter or an environmental condition of the medical device. The environmental parameter may for example be acquired with a sensor of the medical device.

The first defined time interval ZS comprises a period for which the at least one operating parameter is determined from the log data. The first defined time interval ZS may in particular comprise a plurality of disjunctive time blocks ZB01, ZB02, ZB03, ZB04, ZB05. The disjunctive time blocks ZB01, . . . , ZB05 may in particular subdivide the first defined time interval ZS into a plurality of intervals or time intervals. The disjunctive time blocks ZB01, . . . , ZB05 may here follow one another temporally without overlapping or being superimposed on one another. The time blocks ZB01, . . . , ZB05 may in particular all be of equal size. The at least one operating parameter may in particular be determined cumulatively for each time block ZB01, . . . , ZB05. In other words, the operating parameter may be individually determined for each time block ZB01, . . . , ZB05. For example, a number of times or frequency with which a program or a function of the medical device is run can here be summed for a time block ZB01, . . . , ZB05. Alternatively, it is possible to determine an average of the operating parameter over the corresponding time block ZB01, . . . , ZB05 or a list of the operating parameters for the corresponding time block ZB01, . . . , ZB05. Whether it is the sum, the average or a list of the operating parameters which is determined for the corresponding time block ZB01, . . . , ZB05 depends on the nature of the operating parameter or on what the operating parameter describes. A time block ZB01, . . . , ZB05 may for example comprise a week or seven days. In this way, it is for example possible to offset fluctuations in the operating parameter over a weekend, since values for the at least one operating parameter are averaged or summed or listed over a week. Alternatively, a time block ZB01, . . . , ZB05 may for example comprise one month. The first defined time interval ZS may comprise any desired number of time blocks ZB01-ZB05. In particular, the first defined time interval ZS may comprise a time block ZB01, . . . , ZB05. In particular, the first defined time interval ZS may comprise more than one time block ZB01, . . . , ZB05. The first defined time interval ZS may in particular be predetermined or defined by a user. For this purpose, the user may state, for example with the assistance of calendar dates, from when to when the first defined time interval ZS should extend. Alternatively, the user can state a duration which the first defined time interval ZS should comprise. The first defined time interval ZS may here end on the day on which the satisfaction information is generated. The first defined time interval ZS then begins on the day which is determined beginning from the end day, in accordance with the duration of the first defined time interval ZS. Alternatively, the duration of the first defined time interval ZS may be predetermined. The prediction time block VZB may in particular temporally directly follow the first defined time interval ZS.

In the method step DET-02 of determining the at least one item of customer information, the at least one item of customer information is determined based upon sales data and/or customer service data for a second defined time interval ZS.

The sales data may in particular comprise information about a number and/or type of supplied and/or ordered spare parts for the medical device. Alternatively or additionally, the sales data may comprise information as to how many medical devices the customer owns or manages. Alternatively or additionally, the sales data may comprise costs which the customer had already expended in relation to the medical device.

The customer service data may in particular comprise information about the customer. This information may for example be derived from customer surveys. Alternatively or additionally, the customer service data may comprise information about the number and/or urgency and/or type of service tickets which the customer has sent to customer services. The type of service ticket may describe where the service ticket has to be processed, whether it is an inquiry, a complaint or a defect in the medical device etc. Account may here in particular be taken of service tickets which relate to the medical device for which the satisfaction information is to be prepared. Alternatively or additionally, account may be taken of all the customer's service tickets. In particular, account may be taken of open and already resolved service tickets. Alternatively or additionally, customer service data may also be derived from a conversation with the customer. Alternatively or additionally, customer service data may comprise information as to how frequently a technician has already made on-site visits to the customer. Alternatively or additionally, the customer service data information may comprise information about the volume and/or term of a contract and/or further contractual details of the customer.

The second defined time interval ZS is configured similarly to the first defined time interval ZS. In particular, the second defined time interval ZS may correspond to the first defined time interval US. In particular, the at least one item of customer information is determined cumulatively for a time block ZB01, . . . , ZB05. In particular, the customer information may be averaged or summed or listed over the time block ZB01, . . . , ZB05.

Determination DET-01 of the at least one operating parameter and/or determination DET-02 of the at least one item of customer information may be carried out using a feature extraction algorithm. The feature extraction algorithm may for example be prepared by an expert. Alternatively or additionally, the feature extraction algorithm may determine the at least one operating parameter and/or the at least one item of customer information by means of analytical analysis. Alternatively or additionally, the feature extraction algorithm may comprise a second trained function. In some embodiments, the feature extraction algorithm may be part of the first trained function. In particular, the at least one operating parameter may be determined by a first feature extraction algorithm. In particular, the at least one item of customer information may be determined by a second feature extraction algorithm. In particular, the first and second feature extraction algorithms may differ. In particular, the first feature extraction algorithm may comprise a sequence recognition algorithm (sequence mining or sequence pattern mining). In particular, the second feature extraction algorithm may comprise “natural language processing”.

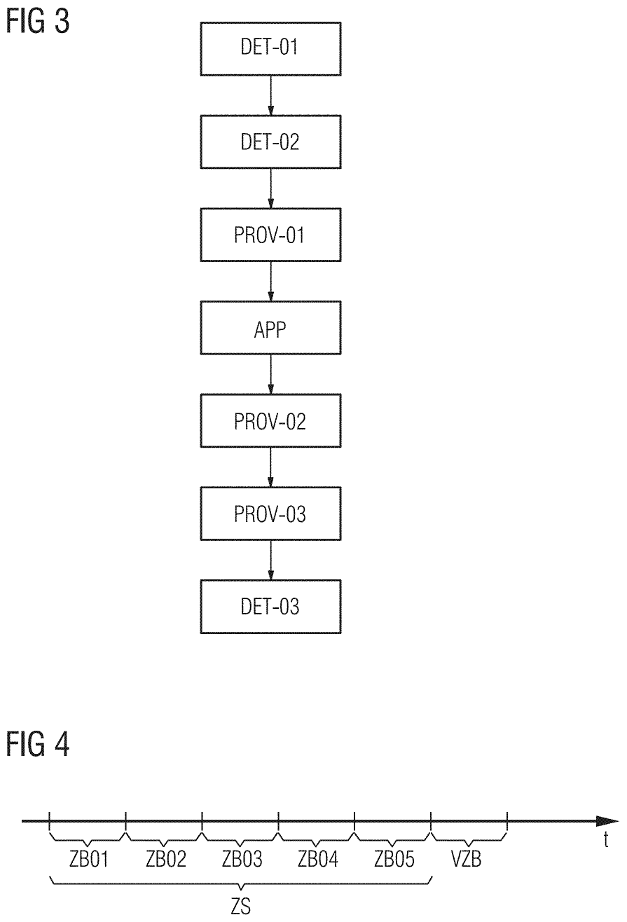

FIG. 3 shows a third example embodiment of a method for providing an item of satisfaction information about a customer's predicted satisfaction with regard to a medical device.

The method steps PROV-01 of providing the input data, APP of applying the first trained function and PROV-02 of providing the satisfaction information are carried out in accordance with the description in relation to FIG. 1. The method steps DET-01 of determining the at least one operating parameter and DET-02 of determining the at least one item of customer information are carried out in accordance with the description in relation to FIG. 2.

In the method step PROV-03 of providing the satisfaction information, the satisfaction information is provided in a decision support system. The satisfaction information is here provided to the user in the decision support system. Provision may in particular proceed by displaying the satisfaction information by a display medium or output medium. The output medium may in particular be a screen or a computer screen. The satisfaction information may be displayed or provided in the decision support system in the form of an image or graphic and/or text. In particular, the decision support system may comprise a graphical user interface (GUI) by means of which the satisfaction information can be represented or displayed.

In the method step DET-03 of deriving a recommended action, the recommended action is derived from satisfaction information by the decision support system. The recommended action may state which measure the user should carry out in order to improve or ensure the customer's satisfaction or to prevent escalation by the customer. The recommended action may for example be a recommendation to make a telephone call, send spare parts, make a customer visit, make an offer to the customer or offer a discount to the customer, to wait, to answer a customer inquiry, etc. Alternatively or additionally, the recommended action may comprise prioritizing a plurality of customers. An item of satisfaction information has been provided in advance according to the inventive method for each customer of the plurality of customers. Depending on this satisfaction information, the plurality of customers can be prioritized. Prioritization indicates which customer should be handled preferentially or particularly quickly or which customer inquiry should be processed in the particularly near future.

The recommended action can be provided in the decision support system. The recommended action can be displayed by means of the display medium of the decision support system. The recommended action make take the form of a graphic or image and/or text.

FIG. 4 shows an example embodiment of a defined time interval ZS comprising a plurality of disjunctive time blocks ZB01, . . . , ZB05 and a prediction time block VZB.

The representation of FIG. 4 explains a time profile with the assistance of the horizontal arrow. “t” here denotes time. The first and second time intervals ZS stated in the description may both be configured according to the defined time interval ZS described here. The defined time interval ZS is here divided into five disjunctive time blocks ZB01, . . . ZB05. The time blocks ZB01, . . . ZB05 here follow one another temporally. The time blocks ZB01, . . . , ZB05 here describe the entire defined time interval ZS. The at least one operating parameter and/or the at least one item of customer information may be determined cumulatively for each time block ZB01, . . . ZB05 as described above. A time profile of the at least one operating parameter and/or of the at least one item of customer information over the defined time interval ZS can thus be determined. This time profile may then in particular serve as input data for the first trained function.

The prediction time block VZB directly follows the defined time interval. In the step APP of applying the first trained function, the satisfaction information for the prediction time block VZB is determined based upon the at least one operating parameter and the at least one item of customer information.

In particular, the defined time interval ZS may be in the past and the prediction time block VZB in the future. In other words, based upon known data (operating parameter, customer information), the customer's satisfaction may be predicted by means of the satisfaction information.

FIG. 5 shows an example embodiment of a method for providing a first trained function.

In the method step TPROV-10 of providing training input data, the training input data is input into a training system. The training input data comprises at least one operating parameter of a medical device and at least one item of customer information. Provision TPROV-01 of the training input data may proceed similarly to the provision PROV-01 of the input data as described in the description in relation to FIG. 1.

In the method step TPROV-02 of providing training output data, the training output data is provided to the training system. The training output data here comprises an item of satisfaction information about the customer's predicted satisfaction with regard to a medical device. The training output data relates to the training input data. For this purpose, the training output data may have been determined by an expert or user based upon the training input data. In particular, the training output data may have been prepared with the assistance of the expert's or user's observations or experience in respect of the training input data. In particular, customer feedback may be taken into account during preparation. Provision TPROV-02 of the training output data may proceed similarly to provision PROV-01 of the input data.

In the method step TRAIN of training the first trained function, the first trained function is trained based upon the training input data and the training output data. In particular, for this purpose the first trained function is trained in such a manner that an item of satisfaction information generated by the first trained function and based on the training input data deviates as little as possible from the associated training output data. This deviation is quantified by a match value.

The method step TRAIN of training the first trained function may be carried out for a plurality of first trained functions.

In the step TPROV-03 of providing the first trained function, the first trained function is provided to the user such that they can use the first trained function in carrying out the method according to the invention for determining the satisfaction information. If a plurality of first trained functions has been trained the step TRAIN of training, the first trained function which has the best match value may be provided.

While the method according to the invention is being carried out, the first trained function or the plurality of first trained functions may be further trained by means of feedback. For this purpose, further training output data for the input data is subsequently generated based upon the user's experience or observation. The further training output data here corresponds to satisfaction ascertained by the user. Alternatively, the user may state the match value between the predicted customer satisfaction in the satisfaction information and an ascertained or observed customer satisfaction. Based upon this match value, the first trained function or the plurality of first trained functions may be continuously further trained while the method according to the invention is being carried out. The input data here serves as training input data. In particular, based upon the match value of this training, another first function from the plurality of first trained functions may be provided if said function proves more suitable on account of the match value.

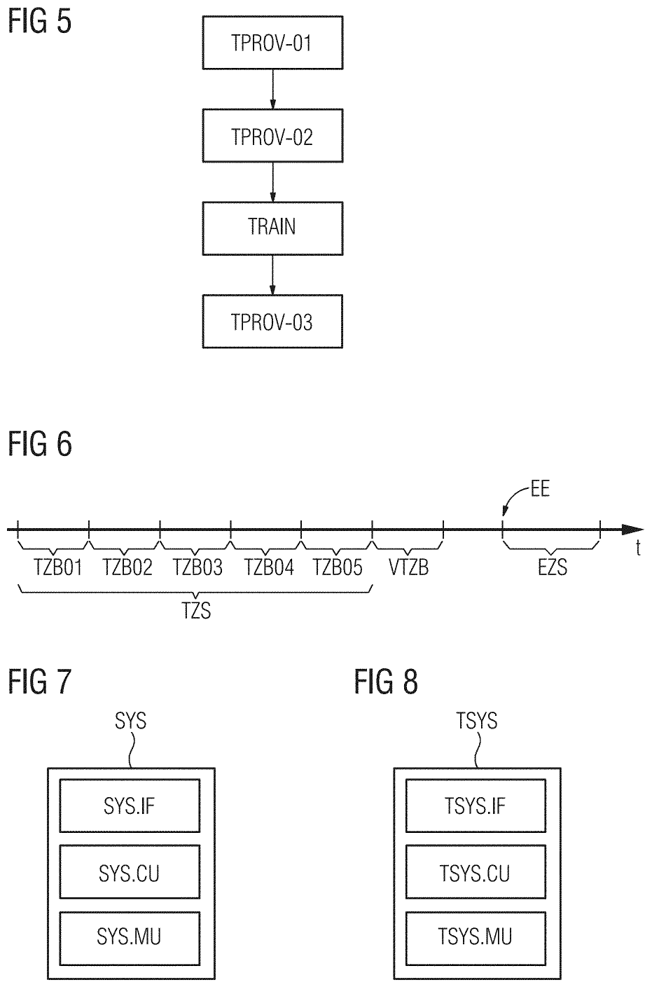

FIG. 6 shows an example embodiment of a training time interval TZS comprising a plurality of disjunctive training time blocks TZB01, TZB02, TZB03, TZB04, TZB05 and a prediction training time interval VTZB, an escalation time interval EZS and an escalation event EE.

The training time interval TZS may be configured similarly to the defined time interval ZS described according to FIG. 4. The disjunctive training time blocks TZB01, . . . , TZB05 may be configured similarly to the disjunctive time blocks ZB01, . . . , ZB05 according to FIG. 4. The prediction training time block VTZB may be configured similarly to the prediction time block VZB according to FIG. 4. However, relative to the training, both the training time interval TZB and the prediction training time block VTZB are located in the past and therefore the training output data can be determined. The training input data is here provided for the training time interval TZS. The training input data may here be configured similarly to the input data for the plurality of disjunctive training time blocks TZB01, . . . , TZB05. The training output data is provided for the prediction training time block VTZB.

The representation moreover shows an escalation event EE and an escalation time interval EZB initiated by the escalation event EE. The escalation event EE may be an event initiated by the customer which indicates major dissatisfaction on the part of the customer. The escalation event may for example be a detailed complaint from the customer or a contract discontinuation or termination. The escalation time interval EZS is the time interval during which the customer's behavior and satisfaction is influenced by the escalation event EE. A duration of the escalation time interval EZS may here depend on the escalation event EE.

The training time interval TZS and the prediction training time block TZB are here located outside the escalation time interval EZS. Any distortion of the training by the escalation event EE may thus be avoided. The escalation event EE and the escalation time interval EZS may be determined and defined by an expert or a user.

In order to generate as much training input data and training output data as possible, the training time interval TZS and the prediction training time interval VTZS may be shifted along the time axis. Training input data and training output data may be generated for different positions. The training time interval TZS and the prediction training time interval VTZS are here located outside the escalation time interval EZS. This shift of the time interval may be made according to a sliding window method.

FIG. 7 shows a system SYS for providing an item of satisfaction information about a customer's predicted satisfaction with regard to a medical device and FIG. 8 shows a training system TSYS for providing a first trained function.

The presented system SYS for providing the satisfaction information is configured to carry out a method according to the invention for providing the satisfaction information about the customer's predicted satisfaction with regard to the medical device. The presented training system TSYS is configured to carry out a method according to the invention for providing the first trained function. The system SYS comprises an interface SYS.IF, a computing unit SYS.CU and a memory unit SYS.MU. The training system TSYS comprises a training interface TSYS.IF, a training computing unit TSYS.CU and a training memory unit TSYS.MU.

The system SYS and/or the training system TSYS may in particular be a computer, a microcontroller or an integrated circuit (IC). Alternatively, the system SYS and/or the training system TSYS may be a real or virtual computer network (a technical name for a real computer network is “cluster” and a technical name for a virtual computer network is “cloud”). The system SYS and/or the training system TSYS may be configured as a virtual system which is run on a computer or a real computer network or a virtual computer (a technical name is “virtualization”).

The interface SYS.IF and/or the training interface TSYS.IF may be a hardware or software interface (e.g. a PCI bus, USB or FireWire). The computing unit SYS.CU and/or the training computing unit TSYS.CU may comprise hardware and/or software components, for example a microprocessor or a field programmable gate array (FPGA). The memory unit SYS.MU and/or the training memory unit TSYS.MU may be configured as a volatile working memory (random access memory, RAM) or as a non-volatile mass storage device (hard disk, USB stick, SD card, solid state disk (SSD)).

The interface SYS.IF and/or the training interface TSYS.IF may in particular comprise a plurality of subinterfaces which carry out different method steps of the respective method according to the invention. In other words, the interface SYS.IF and/or the training interface TSYS.IF may be configured as a plurality of interfaces SYS.IF and/or training interfaces TSYS.IF. The computing unit SYS.CU and/or the training computing unit TSYS.CU may in particular comprise a plurality of subcomputing units which carry out different method steps of the respective method according to the invention. In other words, the computing unit SYS.CU and/or the training computing unit TSYS.CU may be configured as a plurality of computing units SYS.CU and/or training computing units TSYS.CU.

Where it has not yet been explicitly done but is reasonable and in line with the purposes of the invention, individual example embodiments, individual sub-aspects or features thereof may be combined with one another or interchanged without going beyond the scope of the present invention. Advantages of the invention described in relation to one example embodiment also apply, where transferable, to other example embodiments without being explicitly stated to do so.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for providing at least one item of satisfaction information about a predicted satisfaction of a customer regarding to a medical device, the computer-implemented method comprising:
providing input data, the input data including at least one operating parameter of the medical device and at least one item of customer information;
applying a first trained function to the input data, to generate the at least one item of satisfaction information; and
providing the at least one item of satisfaction information.

2. The computer-implemented method of claim 1, further comprising:
determining the at least one operating parameter from log data of the medical device for a first defined time interval.

3. The computer-implemented method of claim 1, further comprising:
determining the at least one item of customer information based upon at least one of sales data and customer service data for a defined time interval.

4. The computer-implemented method of claim 2, wherein at least one of the at least one operating parameter and the at least one item of customer information is determined by a feature extraction algorithm, the feature extraction algorithm including a second trained function.

5. The computer-implemented method of claim 2, wherein the first defined time interval includes a plurality of disjunctive time blocks, the plurality of disjunctive time blocks following one another temporally, and wherein the at least one operating parameter or the at least one item of customer information is determined cumulatively for each of the plurality of disjunctive time blocks.

6. The computer-implemented method of claim 2, wherein the at least one item of satisfaction information is generated for at least one prediction time block, and wherein the at least one prediction time block temporally follows the first defined time interval.

7. The computer-implemented method of claim 1, wherein the at least one item of satisfaction information includes at least one item of classification information.

8. The computer-implemented method of claim 7, wherein the at least one item of satisfaction information includes at least one item of explanatory information about the at least one item of classification information.

9. The computer-implemented method of claim 1, further comprising:
providing the at least one item of satisfaction information in a decision support system, and
deriving a recommended action from the at least one item of satisfaction information by the decision support system.

10. A computer-implemented method for providing a first trained function, the computer-implemented method comprising:
providing training input data, the training input data including at least one operating parameter of a medical device and at least one item of customer information;
providing training output data, the training output data including an item of satisfaction information about predicted satisfaction of a customer with regard to the medical device, and the training output data and the training input data relating to one another;
training the first trained function based upon the training input data and the training output data; and
providing the first trained function after the training.

11. The computer-implemented method of claim 10, wherein the training input data is acquired outside an escalation time interval, the escalation time interval being initiated by an escalation event.

12. The computer-implemented method of claim 10, wherein the first trained function is continuously further trained via feedback, the feedback being based on a match value between the provided satisfaction information and an ascertained customer satisfaction.

13. The computer-implemented method of claim 12, wherein the first trained function is selected from a plurality of first trained functions, selection being based on the match value.

14. A system for providing at least one item of satisfaction information about a predicted satisfaction of a customer with regard to a medical device, the system comprising:
at least one processor configured to
provide input data, the input data including at least one operating parameter of the medical device and at least one item of customer information,
apply a first trained function to the input data, to generate the at least one item of satisfaction information; and
an interface, configured to provide the at least one item of satisfaction information.

15. A non-transitory computer program product storing a computer program, the computer program being directly loadable into a storage device of a system and including program parts for carrying out the method of claim 1 when the program parts are run by the system.

16. A non-transitory computer-readable storage medium storing program parts, readable and runnable by a system to carry out the method of claim 1 when the program parts are run by the system.

17. The computer-implemented method of claim 2, further comprising:
determining the at least one item of customer information based upon at least one of sales data and customer service data for a second defined time interval.

18. The computer-implemented method of claim 3, wherein at least one of the at least one operating parameter and the at least one item of customer information is determined by a feature extraction algorithm, the feature extraction algorithm including a second trained function.

19. The computer-implemented method of claim 17, wherein at least one of the first defined time interval and the second defined time interval includes a plurality of disjunctive time blocks, the plurality of disjunctive time blocks following one another temporally, and wherein the at least one operating parameter or the at least one item of customer information is determined cumulatively for each of the plurality of disjunctive time blocks.

20. The computer-implemented method of claim 17, wherein the at least one item of satisfaction information is generated for at least one prediction time block, and wherein the at least one prediction time block temporally follows at least one of the first defined time interval and the second defined time interval.

21. A non-transitory computer program product storing a computer program, the computer program being directly loadable into a storage device of a system and including program parts for carrying out the method of claim 10 when the program parts are run by the system.

22. A non-transitory computer-readable storage medium storing program parts, readable and runnable by a system to carry out the method of claim 10 when the program parts are run by the system.

23. The computer-implemented method of claim 10, wherein the at least one operating parameter is determined for a first training time interval and the at least one item of customer information for at least one second training time interval.

24. The computer-implemented method of claim 23, wherein the first training time interval and the second training time interval each comprise a plurality of disjunctive training time blocks.
